(19) United States
(12) Patent Application Publication
(10) Pub. No.: US 2026/0224889 A1
(43) Pub. Date: Aug. 6, 2026

(54) ELECTRICAL MUSCLE STIMULATION DEVICE FOR TOILET SEAT, AND ELECTRICAL MUSCLE STIMULATION SYSTEM FOR TOILET SEAT

(71) Applicant: SOCIAREAL CO., LTD, Chiyoda-Ku, Tokyo (JP)

(72) Inventor: Masamichi HAYASHI, Chiyoda-Ku, Tokyo (JP)

(21) Appl. No.: 19/147,935

(22) PCT Filed: Apr. 17, 2024
(86) PCT No.: PCT/JP2024/015204

§ 371 Date: Jul. 14, 2025

(30) Foreign Application Priority Data
Apr. 24, 2023 (JP) 2023-070592

(51) Int. Cl. A61N 1/04; A61N 1/36

(52) U.S. Cl. CPC A61N 1/0472; A61N 1/0452; A61N 1/3603

(57) ABSTRACT

Provided is an electrical muscle stimulation device for toilet seat that is attached to a toilet seat and that applies an electrical muscle stimulation to the lower body of a human body. The electrical muscle stimulation device has a main body including: an electrode that applies electricity to the lower body of a human body; a conductive portion electrically connected to the electrode; and a control unit electrically connected to the conductive portion. The electrode is disposed on an upper surface of the main body, and the main body can be attached to the toilet seat such that the main body does not stick out of the toilet seat when the main body is viewed in a direction perpendicular to the upper surface.

### TECHNICAL FIELD

The present invention relates to an electrical muscle stimulation device for toilet seat, the electrical muscle stimulation device having an electrode that causes a current to flow in a human body to apply an electrical muscle stimulation, and to a toilet seat with electrical muscle stimulation function.

### BACKGROUND ART

As a person gets older, the person has fewer opportunities for exercising and consequently muscles making up the buttocks and legs decrease. It is said that this increases a burden on joints and bones during walking and may lead to difficulty in walking or weaker basal metabolism that accelerates an aging process.

There also are people who fail to do sufficient strength training because of external environmental constraints, such as weather, chronic lack of exercise resulting from a dislike of exercising, excessive obesity, or an orthopedic disease.

In the medical and sports industries, electrical muscle stimulation (which will hereinafter be referred to as EMS) is known, which induces skeletal muscle contraction for muscle reinforcement, incontinence control, spinal malformation control, spasticity control, and the like.

Specifically, a muscle stimulation method by which a muscle is caused to tense and relax, based on an electrical signal outputted from an adhesive pad, has been proposed.

Patent Literature 1 describes a configuration of a clothing-shaped electrode attachment member that transmits an electric signal to quadriceps femoris or hamstrings of a human body to apply an electrical muscle stimulation.

### CITATION LIST

### Patent Literature

Patent Literature 1: Japanese Patent No. 4480797.

### SUMMARY OF INVENTION

### Technical Problem

However, in a case where an electrical muscle stimulation is to be applied to the human body, the electrode attachment member described in Patent Literature 1 requires a troublesome process of taking off clothes wearing, wearing the electrode attachment member, and then changing from the electrode attachment member to the original clothes after the electrical muscle stimulation is applied. This poses a problem that opportunities to apply the electrical muscle stimulation to the human body may decrease gradually.

An object of the present invention for solving the above problem is to, by providing a toilet bowl with an electrode that applies an electrical muscle stimulation, effectively utilize an opportunity to take off clothes from the lower body to save trouble of changing clothes so that an electrical muscle stimulation can be applied daily to a human body.

### Solution to Problem

In order to solve the above problem, the present invention provides an electrical muscle stimulation device for toilet seat that is attached to a toilet seat and that applies an electrical muscle stimulation to a lower body of a human body, the electrical muscle stimulation device having a main body including: an electrode that applies electricity to a lower body of a human body; a conductive portion electrically connected to the electrode; and a control unit electrically connected to the conductive portion, in which the electrode is disposed on an upper surface of the main body, and the main body can be attached to the toilet seat such that the main body does not stick out of the toilet seat when the main body is viewed in a direction perpendicular to the upper surface.

In this configuration, an opportunity to take off clothes from the lower body is utilized effectively to save trouble of changing clothes so that an electrical muscle stimulation can be applied daily to the human body.

(Deleted)

(Deleted)

The electrical muscle stimulation device for toilet seat may have a configuration in which the main body is of an elongated and curved shape, has one end and the other end in a direction in which the body is elongated and extended in a curved manner, and has a first electrode on a side closer to the one end and a second electrode on a side closer to the other end.

In this configuration, an opportunity to take off clothes from the lower body is utilized effectively, so that when a person sits on the toilet seat, an electrical muscle stimulation can be applied to the gluteus maximus muscle and the hamstrings.

The gluteus maximus muscle is the largest muscle in the buttocks. It extends from the back of the pelvis to the side of the thighs to support the upper body, and plays a role of connecting respective movements of the upper body and the lower body. The hamstrings collectively refer to three muscles (biceps femoris, semimembranosus, semitendinosus) present in an area extending from the base of the buttocks to the back side of the thigh and in an area extending from the thigh to the vicinity of the back of the knee. It is said that when the hamstrings are stiff or the hamstrings muscles do not function, a pain in the buttocks or a backache or knee pain is likely to result.

An electrical muscle stimulation system for toilet seat may be configured to include: an electrical muscle stimulation device for toilet seat; a lower attachment portion that allows an electrical muscle stimulation to be applied to the gastrocnemius muscle; a control unit for the lower attachment portion, the control unit giving an instruction to an electrode in the lower attachment portion; and a remote controller that sends at least an instruction to start or stop application of an electric muscle stimulation, to the control unit and to the control unit for the lower attachment portion.

In this configuration, an opportunity to take off clothes from the lower body is utilized effectively, so that when a person sits on the toilet seat, an electrical muscle stimulation can be applied to the gastrocnemius muscle.

The gastrocnemius muscle is a muscle located outside the soleus muscle. The gastrocnemius muscle sticks to the muscle femur, where the tendon of the gastrocnemius muscle and the tendon of the soleus muscle join together to form the Achilles tendon, which sticks to the heel. It is a muscle used for jumping or running motions during exercise. For example, this muscle is used to stand on tiptoes or let the foot strongly push against the ground backward when walking.

The electrical muscle stimulation device for toilet seat may have a configuration in which the main body is of a U shape having a base connecting two front end portions, and the two front end portions each have a first electrode on a front end side and a second electrode on a base side.

In this configuration, an opportunity to take off clothes from the lower body is utilized effectively, so that when a person sits on the toilet seat, an electrical muscle stimulation can be applied to the gluteus maximus muscle and the hamstrings.

The electrical muscle stimulation device for toilet seat may be configured such that a conductive portion electrically connecting the control unit to the first electrode and a conductive portion electrically connecting the control unit to the second electrode are separately provided to allow the conductive portions to perform different controls, respectively.

In this configuration, an electrical muscle stimulation fit for an individual's physical condition can be applied to the human body.

Further, in order to solve the above problem, the present invention provides a toilet seat with electrical muscle stimulation function that applies an electrical muscle stimulation to a human body, the toilet seat including an electrode with which the lower body of a person can come in contact when the person sits on the toilet seat.

In this configuration, an opportunity to take off clothes from the lower body is utilized effectively to save trouble of changing clothes so that an electrical muscle stimulation can be applied daily to the human body.

The toilet seat with electrical muscle stimulation function may have a configuration in which the electrode is disposed at a position at which, when a person sits on the toilet seat, the electrode can apply an electrical muscle stimulation to the gluteus maximus muscle and the hamstrings.

In this configuration, an opportunity to take off clothes from the lower body is utilized effectively, so that when a person sits on the toilet seat, an electrical muscle stimulation can be applied to the gluteus maximus muscle and the hamstrings.

The toilet seat with electrical muscle stimulation function may be configured to include a lower attachment portion having an electrode disposed at a position at which, when a person sits on the toilet seat, the electrode can apply an electrical muscle stimulation to the gastrocnemius muscle.

In this configuration, an opportunity to take off clothes from the lower body is utilized effectively, so that when a person sits on the toilet seat, an electrical muscle stimulation can be applied to the gastrocnemius muscle.

The toilet seat with electrical muscle stimulation function may be configured to include an upper attachment portion having an electrode disposed at a position at which, when a person sits on the toilet seat, the electrode can apply an electrical muscle stimulation to an upper part of the thigh.

In this configuration, an opportunity to take off clothes from the lower body is utilized effectively, so that when a person sits on the toilet seat, an electrical muscle stimulation can be applied to the upper part of the thigh.

The toilet seat with electrical muscle stimulation function may be configured to include a control unit that controls a voltage and a frequency that are applied to the electrode.

In this configuration, an electrical muscle stimulation fit for an individual's physical condition can be applied to the human body.

### Advantageous Effects of Invention

According to the electrical muscle stimulation device for toilet seat and the electrical muscle stimulation system for toilet seat of the present invention, by providing a toilet bowl or the like with an electrode that applies an electrical muscle stimulation, an opportunity to take off clothes from the lower body is utilized effectively to save trouble of changing clothes so that an electrical muscle stimulation can be applied daily to the human body.

### BRIEF DESCRIPTION OF DRAWINGS

FIG. 1 is a perspective view for explaining an electrical muscle stimulation device for toilet seat according to a first embodiment of the present invention.

FIG. 2 shows (a) a front view and (b) a side view for explaining the electrical muscle stimulation device for toilet seat according to the first embodiment of the present invention.

FIG. 3 is a perspective view for explaining an electrical muscle stimulation device for toilet seat according to a second embodiment of the present invention.

FIG. 4 is a perspective view for explaining an electrical muscle stimulation device for toilet seat according to a third embodiment of the present invention.

FIG. 5 is a schematic diagram for explaining a form of use of the electrical muscle stimulation device for toilet seat according to the third embodiment of the present invention.

FIG. 6 is a schematic diagram for explaining a form of use of an electrical muscle stimulation device for toilet seat according to a fourth embodiment of the present invention.

FIG. 7 is a perspective view for explaining a toilet seat with electrical muscle stimulation function according to a fifth embodiment of the present invention.

FIG. 8 is a perspective view for explaining a toilet seat with electrical muscle stimulation function according to a sixth embodiment of the present invention.

### DESCRIPTION OF EMBODIMENTS

### First Embodiment

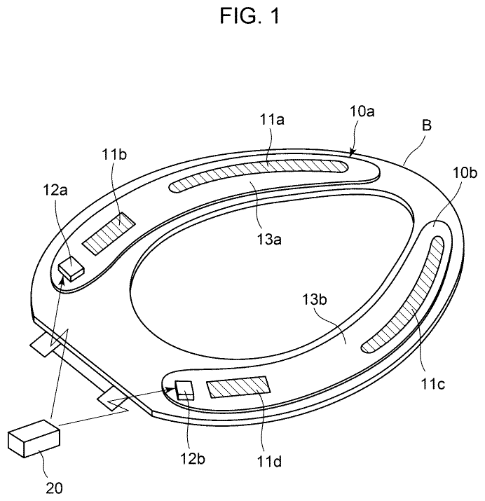

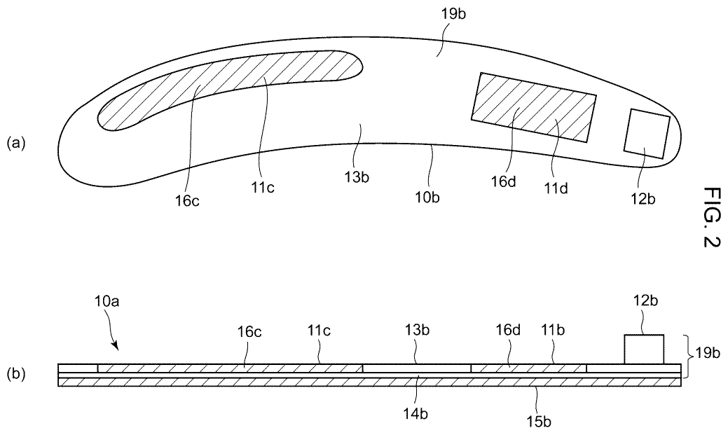

A first embodiment of the present invention will be described with reference to FIGS. 1 and 2. FIG. 1 is a perspective view for explaining an electrical muscle stimulation device for toilet seat according to the first embodiment of the present invention. FIG. 2 shows (a) a front view and (b) a side view for explaining the electrical muscle stimulation device for toilet seat according to the first embodiment of the present invention.

An electrical muscle stimulation device for toilet seat 10 (10a, 10b) according to the first embodiment is used in a state of being attached to a seating surface of a toilet seat B, and includes an electrical muscle stimulation device for toilet seat 10a for the left side of the lower body and an electrical muscle stimulation device for toilet seat 10b for the right side of the lower body. To utilize an opportunity to take off clothes from the lower body, the electrical muscle stimulation device for toilet seat 10a is disposed on and attached to the toilet seat B such that when a person sits on the toilet seat B, an electrode 11 (11a, 11b) comes in contact with the left side of the lower body, and the electrical muscle stimulation device for toilet seat 10b is disposed on and attached to the toilet seat B such that when a person sits on the toilet seat B, an electrode 11(11c, 11d) comes in contact with the right side of the lower body.

The structure of the electrical muscle stimulation device for toilet seat 10 (10a, 10b) will be described by describing the electrical muscle stimulation device for toilet seat 10b for the right side of the lower body, as an example, referring to FIG. 2. An electrode 11c is disposed around the middle of the toilet seat B, as an electrode of a long elliptical shape so that the electrode 11c comes in contact with the hamstrings representing the biceps femoris, the semitendinosus, and the semimembranosus and applies an electrical muscle stimulation to the hamstrings. An electrode 11d is, on the other hand, disposed around a part of toilet seat B that supports the buttocks, as an electrode of a short quadrangular shape so that the electrode 11d comes in contact with the gluteus maximus muscle and applies an electrical muscle stimulation thereto.

In the first embodiment, the electrode 11d is formed into a short quadrangular shape, but, without being limited to this, the configuration may be changed when necessary. For example, the electrode 11d may be formed into a circular or elliptical shape.

In the first embodiment, the electrode 11c for the hamstrings and the electrode 11d for the gluteus maximus muscle are provided, but, without being limited to this, the configuration may be changed when necessary. For example, only the electrode 11d for the gluteus maximus muscle may be provided or only the electrode 11c for the hamstrings may be provided.

Furthermore, the position of each electrode and the number of electrodes may be changed in the electrical muscle stimulation device for toilet seat 10b for the right side of the lower body and in the electrical muscle stimulation device for toilet seat 10a for the left side of the lower body. For example, the electrical muscle stimulation device for toilet seat 10b for the right side of the lower body may be configured to have the electrode 11c only, and the electrical muscle stimulation device for toilet seat 10a for the left side of the lower body may be configured to have the electrode 11b only.

Alternatively, the electrical muscle stimulation device for toilet seat may be configured to include only the electrical muscle stimulation device for toilet seat 10a for the left side of the lower body or only the electrical muscle stimulation device for toilet seat 10b for the right side of the lower body.

As shown in FIG. 2(b), a part of electrode 11 (11c, 11d) that comes in contact with the skin is made of a conductive silicone 16 (16c, 16d) of about 3 mm to 4 mm in thickness, has elasticity, and is made friendly to the skin. The conductive silicone 16 (16c, 16d) is formed by mixing a conductive filler, such as carbon black, into silicone, which is an insulator, to give it conductivity.

Under the conductive silicone 16 (16c, 16d) (on a side closer to the toilet seat B), a metal conductive portion 14b of about 1 mm in thickness is formed in such a way as to be electrically connected to the conductive silicone 16 (16c, 16d) and to be electrically connected to a terminal of a control unit 12b, and is supplied with electricity whose voltage and frequency are determined by the control unit 12b. The electrical muscle stimulation device for toilet seat 10b for the right side of the lower bod is provided with a control unit 12b, and the electrical muscle stimulation device for toilet seat 10a for the left side of the lower body is provided with a control unit 12a similar in configuration to the control unit 12b. This control unit 12a is, however, not illustrated.

The conductive portion 14b can be made of a metal with high conductivity, such as aluminum or copper. In the first embodiment, the conductive portion 14b is connected to a conductive silicone 16c and to a conductive silicone 16d. However, the conductive portion 14b may be divided into a conductive portion connected to the conductive silicone 16c only and a conductive portion connected to the conductive silicone 16d only, and different voltages and frequencies may be applied to these conductive portions, respectively.

A surface area of the electrical muscle stimulation device for toilet seat 10b, the surface area not including the electrode 11 (11c, 11d), serves as an insulating portion 13b of about 3 mm to 4 mm in thickness, is made of synthetic resin, rubber, fiber, or the like that does not conduct electricity, and is bonded to the conductive portion 14b.

Under the insulating portion 13b and the conductive portion 14b (on the side closer to the toilet seat B), a toilet seat attachment portion 15b is bonded, which is a low-adhesive sheet that is identical in size and shape with the electrical muscle stimulation device for toilet seat 10b and that is about 0.5 mm in thickness. With this toilet seat attachment portion 15b that is the low-adhesive sheet, the electric muscle stimulation device for toilet seat 10 (10a, 10b) can be easily pasted to the seating surface of the toilet seat and used, and after being used, can be easily removed and carried away. That is, the electrical muscle stimulation device for toilet seat 10 (10a, 10b) can be attached/detached to/from the toilet seat B freely.

In the present application, the electrode 11 (11a, 11b), a control unit 12, an insulating portion 13 (13a, 13b), and a conductive portion 14 (14a, 14b), which do not include a toilet seat attachment portion 15 (15a, 15b), constitute a main body 19 (19a, 19b).

In the first embodiment, the toilet seat attachment portion 15 is provided as the low-adhesive sheet, but, without being limited to this, the configuration may be changed when necessary. For example, the toilet seat attachment portion may be provided as a band with which the main body 19 (19a, 19b) is wound and attached to the toilet seat B, or the main body 19 (19a, 19b) may be clipped onto the toilet seats B, using a clip-like means. In another case, with no toilet seat attachment portion 15 provided, the main body 19 may be wound around the thigh or the like, using a band or the like. What is needed is that the main body can be disposed such that when a person sits on the toilet seat, at least the electrode comes in contact with the lower body of the person.

On a part of the surface of the electrical muscle stimulation device for toilet seat 10b, the part being close to the buttocks, the control unit 12 (12a, 12b) is disposed. The control unit 12 (12a, 12b) has a function of receiving an instruction from a remote controller 20, which will be described later, via infrared rays, and applying electricity with an adjusted voltage and frequency from a terminal (not illustrated) to the electrode 11(11a, 11b, 11c, 11d), and has a built-in battery that drives the control unit 12 (12a, 12b). A frequency for electrical muscle stimulation application is a low frequency, and is given as a frequency that can be changed in a range of 10 Hz to 50 Hz. However, when letting a stimulation go inside the human body is difficult, the stimulation may be allowed to go deep inside the human body by using interference waves.

The remote controller 20 can control the start and stoppage of an electrical muscle stimulation, and can send an instruction to the control unit 20 via infrared rays to instruct the control unit 12 (12a, 12b) to start or stop the electrical muscle stimulation. In addition, the remote controller 20 has a function of specifying a voltage and a frequency to be applied to the electrode 11 (11a, 11b, 11c, 11d), and has a function of sending the specified voltage and frequency to the control unit 20 via infrared rays. Further, the remote controller 20 controls a remaining time for electrical muscle stimulation application, and has a display unit that displays the remaining time. The remote controller 20 has a built-in battery serving as a power supply needed. In addition, the remote controller 20 may be provided with a storage unit that stores an electrical muscle stimulation use history at each part.

In the first embodiment, the control unit 12 has the built-in battery, but, without being limited to this, the configuration may be changed when necessary. For example, the control unit 12 may have a conversion function of converting a voltage from a commercial power supply into a low voltage, so that a voltage from the commercial power supply, such as 100 V AC, is supplied.

In the first embodiment, the remote controller 20 and the control unit 12 (12a, 12b) communicate with each other via infrared rays, but, without being limited to this, the configuration may be changed when necessary. For example, wireless communication via radio waves may be adopted.

As described above, according to the first embodiment, an electrical muscle stimulation device for toilet seat that is attached to a toilet seat and that applies an electrical muscle stimulation to a human body is provided. The electrical muscle stimulation device for toilet seat includes a main body having an electrode that applies electricity to a human body, and the main body can be disposed such that when a person sits on the toilet seat, the electrode comes in contact with the lower body of the person. By using this electrical muscle stimulation device for toilet seat, an opportunity to take off clothes from the lower body is utilized effectively to save trouble of changing clothes so that an electrical muscle stimulation can be applied daily to the human body.

### Second Embodiment

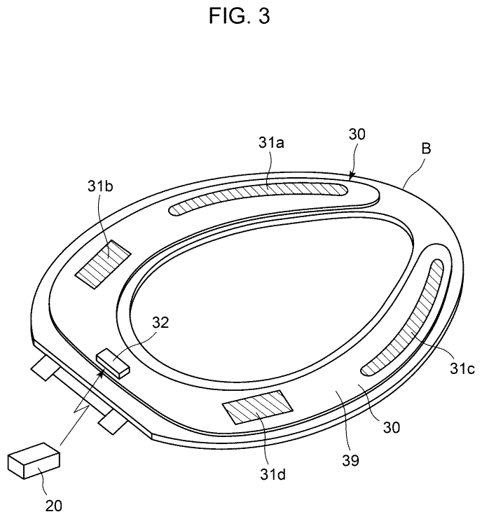

A second embodiment of the present invention is different from the first embodiment in that an electrical muscle stimulation device for toilet seat is not divided into a unit for the left side of the lower body and a unit for the right side of the lower body but is configured into an integral structure. The second embodiment will be described with reference to FIG. 3. FIG. 3 is a perspective view for explaining an electrical muscle stimulation device for toilet seat according to the second embodiment of the present invention.

As shown in FIG. 3, an electrical muscle stimulation device for toilet seat 30 of the second embodiment is not divided into a unit for the left side of the lower body and a unit for the right side of the lower body but is configured into an integral U shape. As a result, a single control unit 32 can control electrical muscle stimulations to the left side of the lower body and electrical muscle stimulations to the right side of the lower body.

Electrodes are disposed in the same manner as in the first embodiment, that is, electrodes 31a and 31c are each disposed around the middle of the toilet seat B, as an electrode of a long elliptical shape so that the electrode comes in contact with the hamstrings representing the biceps femoris, the semitendinosus, and the semimembranosus and applies an electrical muscle stimulation to the hamstrings. Electrodes 31b and 31d are each disposed around a part of toilet seat B that supports the buttocks, as an electrode of a short quadrangular shape so that the electrode comes in contact with the gluteus maximus muscle and applies an electrical muscle stimulation thereto.

Following an instruction from the remote controller 20, the control unit 32 supplies electricity with a given voltage and frequency to electrodes 31 (31a, 31b, 31c, 31d), thus allowing each of them to apply an electrical muscle stimulation.

At this time, electricity supplied to the electrodes 31 (31a, 31b, 31c, 31d) may be identical in voltage and frequency or may be different in voltage and frequency from each other.

In the second embodiment, a main body 39 is attached to the toilet seat B by the toilet seat attachment portion made of the low-adhesive sheet, can be removed easily from the toilet seat B, that is, can be attached/detached to/from the toilet seat B freely, and is therefore can be carried around conveniently.

As described above, according to the second embodiment, an electrical muscle stimulation device for toilet seat that is attached to a toilet seat and that applies an electrical muscle stimulation to a human body is provided. The electrical muscle stimulation device for toilet seat includes a main body having an electrode that applies electricity to a human body, and the main body can be disposed such that when a person sits on the toilet seat, the electrode comes in contact with the lower body of the person. By using this electrical muscle stimulation device for toilet seat, an opportunity to take off clothes from the lower body is utilized effectively to save trouble of changing clothes so that an electrical muscle stimulation can be applied daily to the human body.

### Third Embodiment

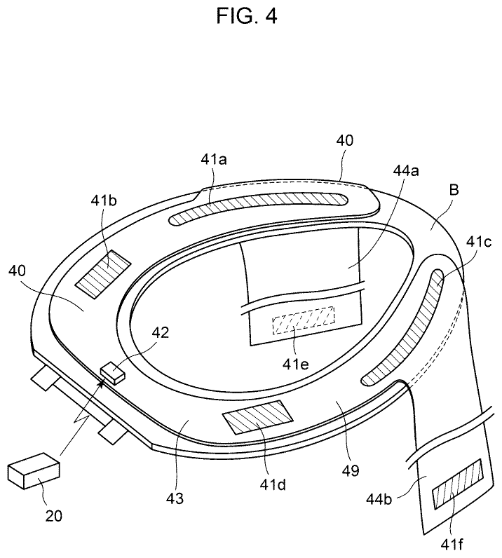

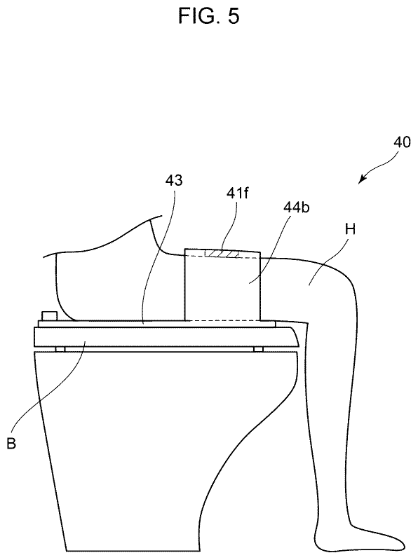

A third embodiment of the present invention is different from the first embodiment and the second embodiment in that an electrical muscle stimulation can be applied also to an upper part (front side) of the thigh. The third embodiment will be described with reference to FIGS. 4 and 5. FIG. 4 is a perspective view for explaining an electrical muscle stimulation device for toilet seat according to the third embodiment of the present invention. FIG. 5 is a schematic diagram for explaining a form of use of the electrical muscle stimulation device for toilet seat according to the third embodiment of the present invention.

In the electrical muscle stimulation device for toilet seat 40 according to the third embodiment, an electrodes 41a for the left side of the lower body and an electrodes 41c for the right side of the lower body are each disposed around the middle of the toilet seat B, as an electrode of a long elliptical shape so that the electrode comes in contact with the hamstrings representing the biceps femoris, the semitendinosus, and the semimembranosus and applies an electrical muscle stimulation to the hamstrings, while an electrodes 41b for the left side of the lower body and an electrodes 41d for the right side of the lower body are each disposed around the part of toilet seat B that supports the buttocks, as an electrode of a short quadrangular shape so that the electrode comes in contact with the gluteus maximus muscle and applies an electrical muscle stimulation thereto. In addition, an electrode 41e for the thigh of the left leg of a human body H and an electrode 41f for the thigh of the right leg are disposed on an upper attachment portion 44a for the left leg of a belt shape and an upper attachment portion 44b for the right leg of a belt shape, respectively, the upper attachment portion 44a and upper attachment portion 44b extending from a main body 49.

The upper attachment portion 44a for the left leg and the upper attachment portion 44b for the right leg are wound around the thigh of the left leg and the thigh of the right leg of the human body H, respectively, and are used in such a manner as shown in FIG. 5. At this time, the positions of the electrodes 41e and 41f are adjusted in such a way as to keep each of the electrodes 41e and 41f in contact with an upper part of the thigh of the leg. Then, electricity with a given voltage and frequency is supplied to the electrode 41e and the electrode 41f, which allows the electrode 41e and the electrode 41f to apply electrical muscle stimulations to upper parts of the thighs of both legs, respectively.

In the third embodiment, the upper attachment portion 44a for the left leg and the upper attachment portion 44b for the right leg are of the belt shapes extending from the main body 49, but, without being limited to this, the configuration may be changed when necessary. For example, a lower attachment portion, which will be described later, may be provided as a separate belt shape not extending from the main body 49.

In the third embodiment, the electrode 41e and the electrode 41f, which apply an electrical muscle stimulation, are disposed on the upper attachment portion 44a for the left leg and the upper attachment portion 44b for the right leg, respectively, but, without being limited to this, the configuration may be changed when necessary. For example, an electrode that applies an electrical muscle stimulation may be disposed only on the upper attachment portion 44a for the left leg or only on the upper attachment portion 44b for the right leg, or control may be performed in such a way as to supply the upper attachment portion 44a for the left leg and the upper attachment portion 44b for the right leg with electricity different in voltage and frequency from each other to apply different electrical muscle stimulations.

As described above, according to the third embodiment, an electrical muscle stimulation device for toilet seat that is attached to a toilet seat and that applies an electrical muscle stimulation to a human body is provided. The electrical muscle stimulation device for toilet seat includes a main body having an electrode that applies electricity to a human body, and the main body can be disposed such that when a person sits on the toilet seat, the electrode comes in contact with the lower body of the person. By using this electrical muscle stimulation device for toilet seat, an opportunity to take off clothes from the lower body is utilized effectively to save trouble of changing clothes so that an electrical muscle stimulation can be applied daily to the human body.

### Fourth Embodiment

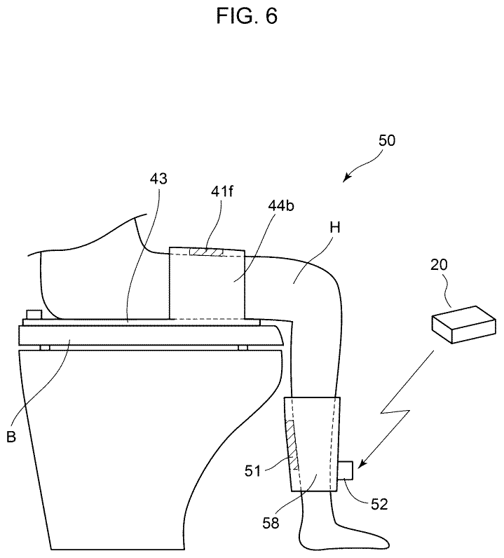

A fourth embodiment of the present invention is different from the first to third embodiments in that an electrical muscle stimulation is applied also to the gastrocnemius muscle. The fourth embodiment will be described with reference to FIG. 6. FIG. 6 is a schematic diagram for explaining a form of use of an electrical muscle stimulation device for toilet seat according to the fourth embodiment of the present invention.

An electrical muscle stimulation device for toilet seat 50 of the fourth embodiment includes an electrode for the hamstrings, an electrode for the gluteus maximus muscle, and an electrode for the upper part (front side) of the thigh, and additionally includes a lower attachment portion 58 (58a, 58b) of a belt shape having an electrode 51 (51a, 51b) that applies an electrical muscle stimulation to the gastrocnemius muscle. The lower attachment portion 58 includes a lower attachment portion 58a for the left leg and a lower attachment portion 58b for the right leg. As shown in FIG. 6, when used, an electrode 51a disposed on the lower attachment portion 58a is positioned in such a way as to be in contact with the gastrocnemius muscle of the left leg and an electrode 51b disposed on the lower attachment portion 58b is positioned in such a way as to be in contact with the gastrocnemius muscle of the right leg, and the lower attachment portions 58 (58a, 58b) of the belt shapes are wound around the left leg and the right leg, respectively.

Then, the remote controller 20 sends a voltage and a frequency to a control unit 52, which receives the voltage and the frequency. Hence a given electrical muscle stimulation can be applied to respective gastrocnemius muscles of the left and right legs. At this time, voltages and frequencies applied to the left and right gastrocnemius muscles may be the same or may be different from each other.

In the fourth embodiment, the lower attachment portion 58 (58a, 58b) is of the belt shape, but, without being limited to this, the configuration may be changed when necessary. For example, a pad shape slightly larger than the electrode 51 (51a, 51b) may be adopted. This lower attachment portion of the pad shape may be pasted to the leg, using a low-adhesive sheet, or may be fixed with a belt.

As described above, according to the fourth embodiment, an electrical muscle stimulation device for toilet seat that is attached to a toilet seat and that applies an electrical muscle stimulation to a human body is provided. The electrical muscle stimulation device for toilet seat includes a main body having an electrode that applies electricity to a human body, and the main body can be disposed such that when a person sits on the toilet seat, the electrode comes in contact with the lower body of the person. By using this electrical muscle stimulation device for toilet seat, an opportunity to take off clothes from the lower body is utilized effectively to save trouble of changing clothes so that an electrical muscle stimulation can be applied daily to the human body.

### Fifth Embodiment

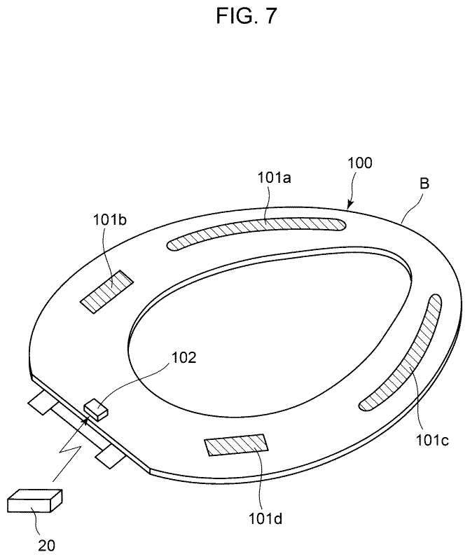

A fifth embodiment of the present invention is different from the first to fourth embodiments in that an electrode is undetachably embedded in the toilet seat to form a toilet seat with electrical muscle stimulation function. The fifth embodiment will be described with reference to FIG. 7. FIG. 7 is a perspective view for explaining a toilet seat with electrical muscle stimulation function according to the fifth embodiment of the present invention.

A toilet seat with electrical muscle stimulation function 100 has an electrode 101 (101a, 101b, 101c, 101d) embedded in the toilet seat B in such a way as to be exposed on the surface of the toilet seat B. An electrode 101a is disposed around the middle of the left side of the toilet seat B, as an electrode of a long elliptical shape so that the electrode comes in contact with the hamstrings of the left side of the lower body, the hamstrings representing the biceps femoris, the semitendinosus, and the semimembranosus, and applies an electrical muscle stimulation to the hamstrings. An electrode 101b is disposed around a part of the left side of toilet seat B that supports the buttocks, as an electrode of a short quadrangular shape so that the electrode comes in contact with the gluteus maximus muscle of the left side of the lower body and applies an electrical muscle stimulation to the gluteus maximus muscle.

An electrode 101c is disposed around the middle of the right side of the toilet seat B, as an electrode of a long elliptical shape so that the electrode comes in contact with the hamstrings of the right side of the lower body, the hamstrings representing the biceps femoris, the semitendinosus, and the semimembranosus, and applies an electrical muscle stimulation to the hamstrings. An electrode 101d is disposed around a part of the right side of toilet seat B that supports the buttocks, as an electrode of a short quadrangular shape so that the electrode comes in contact with the gluteus maximus muscle of the right side of the lower body and applies an electrical muscle stimulation to the gluteus maximus muscle.

The electrode 101 (101a, 101b, 101c, 101d) is made of a conductive silicone, has elasticity, and is made friendly to the skin. The conductive silicone is formed by mixing a conductive filler, such as carbon black, into silicone, which is an insulator, to give it conductivity.

At the rear end of the part of toilet seat B that supports the buttocks, a control unit 102 is disposed, which is connected to the electrode 101(101a, 101b, 101c, 101d). Based on voltage and frequency data received from the remote controller 20, electricity is supplied to the electrode 101 (101a, 101b, 101c, 101d), which applies an electrical muscle stimulation. The control unit 102 can be driven with commercial power, such as a voltage of 100 V, that is supplied through a cord (not illustrated) plugged in a wall outlet. The remote controller 20 has a built-in battery.

When electricity is supplied from the control unit 102 to the electrodes 101 (101a, 101b, 101c, 101d), electricity identical in voltage and frequency may be supplied uniformly or electricity different in voltage and frequency may be supplied separately to each of the electrodes 101 (101a, 101b, 101c, 101d).

The toilet seat B of the fifth embodiment may be made of synthetic resin or ceramic. Any given insulating material can be selected for the toilet seat B.

As described above, according to the fifth embodiment, a toilet seat with electrical muscle stimulation function that applies an electrical muscle stimulation to a human body is provided. The toilet seat with electrical muscle stimulation function includes an electrode disposed in the toilet seat, the electrode applying electricity to the lower body of a person when the person sits on the toilet seat. By using this toilet seat with electrical muscle stimulation function, an opportunity to take off clothes from the lower body is utilized effectively to save trouble of changing clothes so that an electrical muscle stimulation can be applied daily to the human body.

### Sixth Embodiment

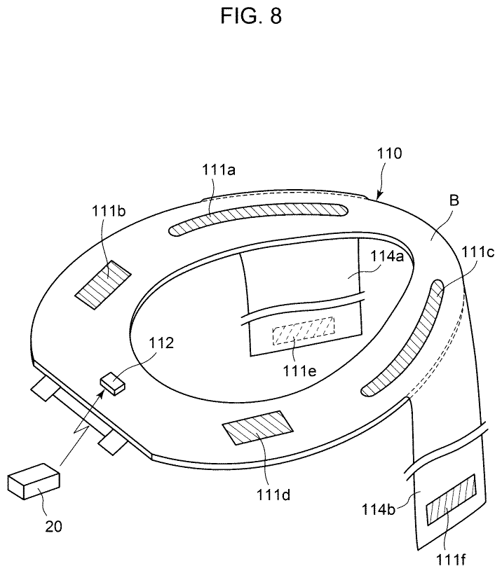

A sixth embodiment of the present invention is different from the fifth embodiment in that an electrical muscle stimulation can be applied also to the upper part (front side) of the thigh. The sixth embodiment will be described with reference to FIG. 8. FIG. 8 is a perspective view for explaining a toilet seat with electrical muscle stimulation function according to the sixth embodiment of the present invention.

A toilet seat with electrical muscle stimulation function 110 of the sixth embodiment includes: an electrode 111a that comes in contact with the hamstrings of the left side of the lower body, the hamstrings representing the biceps femoris, the semitendinosus, and the semimembranosus, and that applies an electrical muscle stimulation to the hamstrings; an electrode 111b that comes in contact with the gluteus maximus muscle of the left side of the lower body and that applies an electrical muscle stimulation to the gluteus maximus muscle; an electrode 111c that comes in contact with the hamstrings of the right side of the lower body, the hamstrings representing the biceps femoris, the semitendinosus, and the semimembranosus, and that applies an electrical muscle stimulation to the hamstrings; and an electrode 111d that comes in contact with the gluteus maximus muscle of the right side of the lower body and that applies an electrical muscle stimulation to the gluteus maximus muscle, and additionally includes: an electrode 111e that applies an electrical muscle stimulation to an upper part (front side) of the left thigh; and an electrode 111f that applies an electrical muscle stimulation to an upper part (front side) of the right thigh.

The electrode 111e is disposed on an upper attachment portion 114a of a belt shape attached to the left side of the toilet seat B, and the electrode 111f is disposed on an upper attachment portion 114b of a belt shape attached to the right side of the toilet seat B. These upper attachment portions 114a and 114b are wound around upper parts of the thighs of the left and right legs of the human body H, respectively, and are used in the manner shown in FIG. 5. At this time, the positions of the electrodes 114e and 114f are adjusted in such a way as to keep each of the electrodes 114e and 114f in contact with the upper part of the thigh of the leg. Hence an electrical muscle stimulation can be applied to respective upper parts of thighs of both legs. Each of the upper attachment portion 114a of the belt shape and the upper attachment portion 114b of the belt shape may be configured such that its end is fixed to the toilet seat B or it can be attached/detached to/from the toilet seat B freely.

When they are configured to be attachable/detachable, cables leading to the electrodes 111e and 111f may be relayed with a connector. The upper attachment portion 114a and the upper attachment portion 114b may not be the belt shapes but be pad shapes slightly larger than the electrodes 111e and 111f, respectively, in which case they are each wound around the leg with a belt or pasted to the leg, using a low-adhesive sheet.

In addition, as described in the fourth embodiment, a lower attachment portion of a belt shape having an electrode that applies an electrical muscle stimulation to the gastrocnemius muscle may also be provided.

As described above, according to the sixth embodiment, a toilet seat with electrical muscle stimulation function that applies an electrical muscle stimulation to a human body is provided. The toilet seat with electrical muscle stimulation function includes an electrode disposed in the toilet seat, the electrode applying electricity to the lower body of a person when the person sits on the toilet seat. By using this toilet seat with electrical muscle stimulation function, an opportunity to take off clothes from the lower body is utilized effectively to save trouble of changing clothes so that an electrical muscle stimulation can be applied daily to the human body.

### Industrial Applicability

The electrical muscle stimulation device for toilet seat and the toilet seat with electrical muscle stimulation function according to the present invention can be widely applied to fields where daily application of an electrical muscle stimulation to the human body is required.

### Reference Signs List

10 (10a, 10b): electrical muscle stimulation device for toilet seat
11 (11a, 11b, 11c, 11d): electrode
12(12a , 12b): control unit
13 (13a, 13b): insulating portion
14 (14a, 14b): conductive portion
15 (15a, 15b): toilet seat attachment portion
16 (16a, 16b, 16c, 16d): conductive silicone
19: main body
20: remote controller
30: electrical muscle stimulation device for toilet seat
31 (31a, 31b, 31c, 31d): electrode
32: control unit
39: main body
40: electrical muscle stimulation device for toilet seat
41 (41a, 41b, 41c, 41d, 41e, 41f): electrode
42: control unit
43: insulating portion
44 (44a, 44b): upper attachment portion
49: main body
50: electrical muscle stimulation device for toilet seat
51: electrode
52: control unit
58: lower attachment portion
100: toilet seat with electrical muscle stimulation function
101 (101a, 101b, 101c, 101d): electrode
102: control unit
110: toilet seat with electrical muscle stimulation function
111 (111a, 111b, 111c, 111d, 111e, 111f): electrode
112: control unit
114 (114a, 114b): upper attachment portion

1. An electrical muscle stimulation device for toilet seat that is attached to a toilet seat and that applies an electrical muscle stimulation to a lower body of a human body, the electrical muscle stimulation device comprising a main body including:
an electrode that applies electricity to a lower body of a human body;
a conductive portion electrically connected to the electrode; and
a control unit electrically connected to the conductive portion,
wherein the electrode is disposed on an upper surface of the main body, and the main body is attached to the toilet seat such that the main body does not stick out of the toilet seat when the main body is viewed in a direction perpendicular to the upper surface.

2. The electrical muscle stimulation device for toilet seat according to claim 1, wherein the main body is of an elongated and curved shape, has one end and the other end in a direction in which the main body is elongated and extended in a curved manner, and has a first electrode on a side closer to the one end and a second electrode on a side closer to the other end.

3. An electrical muscle stimulation system for toilet seat comprising:
the electrical muscle stimulation device for toilet seat according to claim 1;
a lower attachment portion that allows an electrical muscle stimulation to be applied to a gastrocnemius muscle;
a control unit for the lower attachment portion, the control unit giving an instruction to an electrode in the lower attachment portion; and
a remote controller that sends at least an instruction to start or stop application of an electric muscle stimulation, to the control unit and to the control unit for the lower attachment portion.

4. The electrical muscle stimulation device for toilet seat according to claim 1, wherein the main body is of a U shape having a base connecting two front end portions, and the two front end portions each have a first electrode on a front end side and a second electrode on a base side.

5. The electrical muscle stimulation device for toilet seat according to claim 2, wherein a conductive portion electrically connecting the control unit to the first electrode and a conductive portion electrically connecting the control unit to the second electrode are separately provided to allow the conductive portions to perform different controls, respectively.

6. An electrical muscle stimulation system for toilet seat comprising:
the electrical muscle stimulation device for toilet seat according to claim 2;
a lower attachment portion that allows an electrical muscle stimulation to be applied to a gastrocnemius muscle;
a control unit for the lower attachment portion, the control unit giving an instruction to an electrode in the lower attachment portion; and
a remote controller that sends at least an instruction to start or stop application of an electric muscle stimulation, to the control unit and to the control unit for the lower attachment portion.

7. The electrical muscle stimulation device for toilet seat according to claim 4, wherein a conductive portion electrically connecting the control unit to the first electrode and a conductive portion electrically connecting the control unit to the second electrode are separately provided to allow the conductive portions to perform different controls, respectively.
